(19) United States
(12) Patent Application Publication
(10) Pub. No.: US 2025/0120630 A1
(43) Pub. Date: Apr. 17, 2025

(54) Dried Blood Spot Collection Card for Objective Dried Blood Spot Collection Device

(71) Applicant: Blackfly Investments, LLC, DBA Molecular Testing Labs, Vancouver (CA)

(72) Inventors: Peter Flynn, Camas, WA (US); Adam Blackwell, Vancouver, WA (US); Charles Sailey, Vancouver, WA (US)

(21) Appl. No.: 18/380,030

(22) Filed: Oct. 13, 2023

(51) Int. Cl. A61B 5/15

(52) U.S. Cl. CPC A61B 5/150358; A61B 5/150755

(57) ABSTRACT

A dried blood spot collection (DBS) card that assists with correct and efficient collection of capillary blood using a dried blood spot collection (DBS) collection device. The DBS card may comprise a rectangular shape configured to be held in a DBS collection device in a desired orientation for blood sample collection. The DBS card comprises target spots aligned with the collection apertures on the DBS collection device. The patient will be able to direct drops of capillary blood to pass through the top holes and then blood will travel into the DBS card at precise locations so as to ensure proper saturation while minimizing risk of outside contaminants and/or cross-contamination between target spots. Further, the target spots are physically separated with slits or partitions such that the patient can verify saturation level by looking through observation orifices.

### BACKGROUND

As techniques and methodologies for medical testing and diagnostics advance, more and more consumers are turning to remote sample collection using “at-home” test kits that may involve collecting a blood sample using a Dried Blood Spot (DBS) card having the means to collect, store, and ship blood samples from patients to remote diagnostics center for further testing and analysis. In a typical procedure, a patient will induce a blood flow from a finger by pricking the finger allowing blood to flow for enough time to drip onto a dedicated collection medium, such as a DBS card. As is known in the profession, DBS cards may have a typical form factor wherein a patient may deposit up to five blood samples, or more, in dedicated sample areas and then, after allowing time to dry, package the entire DBS card for mailing or transit to a remote testing facility. Results of testing or diagnostics may then be communicated to the patient using standard confidentiality protocols.

However, the standard DBS card form factor is prone to problems such as contamination or spoiled samples. That is, as the portion of the DBS card that collects the blood sample is open to human touching and foreign objects during the collection and transit procedures, the blood sample itself may be compromised to the point of causing unreliable testing results. Further, as the DBS card typically has five “spots” available for blood collection, often times, a typical finger prick may not provide enough blood to fill all five spots. This prompts the patient to collect from another finger after some time. If a blood collection spot dries before being completed or has additional samples to it after some drying has occurred, the entire sample may also be compromised. As is standard with self-collection test kits, collection should be prompt and distinct for each of the typical five spots in order to maximize the chances for proper and reliable testing and diagnostics. Thus, conventional open faced DBS cards are susceptible to opportunities for failure during blood collection and transit.

### BRIEF DESCRIPTION OF THE DRAWINGS

Embodiments of the subject matter disclosed herein in accordance with the present disclosure will be described with reference to the drawings, in which:

FIG. 1 is a top-view diagram of a dried blood spot collection card for use with an objective dried blood spot collection device according to an embodiment of the subject matter disclosed herein;

FIG. 2 is an isometric diagram of an objective dried blood spot collection device according to an embodiment of the subject matter disclosed herein;

FIG. 3 is a diagram view of the top side of the objective dried blood spot collection device of FIG. 2 according to an embodiment of the subject matter disclosed herein;

FIG. 4 is a diagram view of the bottom side of the objective dried blood spot collection device of FIG. 2 according to an embodiment of the subject matter disclosed herein;

FIG. 5 is a diagram view of the one narrow side of the objective dried blood spot collection device of FIG. 2 according to an embodiment of the subject matter disclosed herein;

FIG. 6 is a diagram view of another narrow side of the objective dried blood spot collection device of FIG. 2 according to an embodiment of the subject matter disclosed herein;

FIG. 7 is a diagram view of the objective dried blood spot collection device of FIG. 2 being used in a process for collecting capillary blood from a human according to an embodiment of the subject matter disclosed herein;

FIG. 8 is a diagram view of the objective dried blood spot collection device of FIG. 2 split into two components according to an embodiment of the subject matter disclosed herein;

FIG. 9 is a diagram view of another embodiment of the top side of the objective dried blood spot collection device of FIG. 2 according to an embodiment of the subject matter disclosed herein;

FIG. 10 is a diagram view of yet another embodiment of the top side of the objective dried blood spot collection device of FIG. 2 according to an embodiment of the subject matter disclosed herein; and

FIG. 11 is a top-view diagram of another dried blood spot collection card for use with an objective dried blood spot collection device according to an embodiment of the subject matter disclosed herein.

Note that the same numbers are used throughout the disclosure and figures to reference like components and features.

### DETAILED DESCRIPTION

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

Embodiments will be described more fully hereinafter with reference to the accompanying drawings, which form a part hereof, and which show, by way of illustration, exemplary embodiments by which the devices described herein may be practiced. These devices may, however, be embodied in many different forms and should not be construed as limited to the embodiments set forth herein; rather, these embodiments are provided so that this disclosure will satisfy the statutory requirements and convey the scope of the subject matter to those skilled in the art.

By way of an overview, the systems and devices discussed herein may be directed to a dried blood spot (DBS) card used in conjunction with an objective DBS collection device that assists with correct and efficient collection of capillary blood onto the DBS card. The DBS card may comprise a rectangular shape configured to be held in a DBS collection device in a desired orientation for blood sample collection. The DBS card comprises target spots aligned with the collection apertures on the DBS collection device. The patient will be able to direct drops of capillary blood to pass through the top holes and then blood will travel into the DBS card at precise locations so as to ensure proper saturation while minimizing risk of outside contaminants and/or cross-contamination between target spots. Further, the target spots are physically separated with slits or partitions such that the patient can verify saturation level by looking through observation orifices.

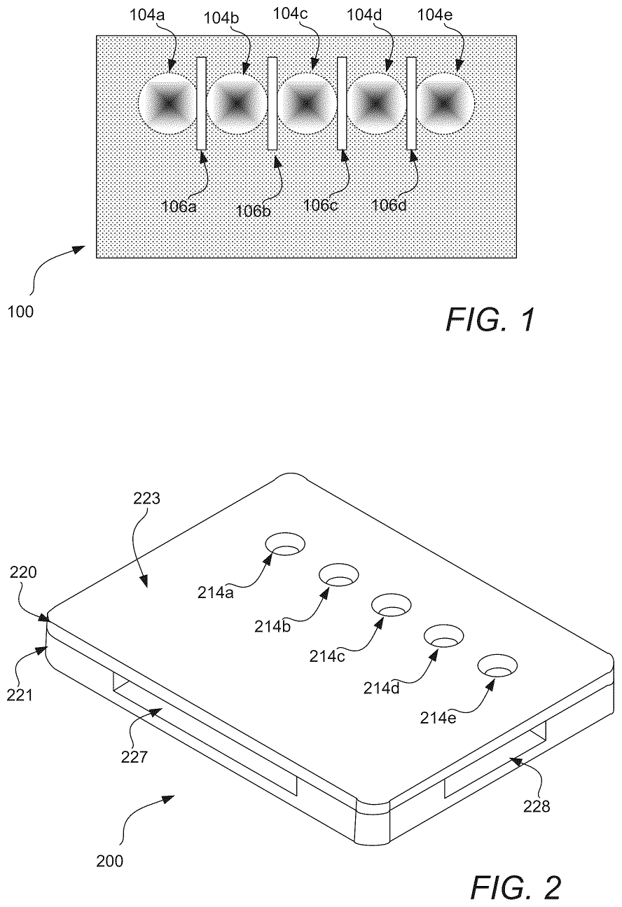

This collection assistance device is an improvement to the collection because the device prevents the patient from rubbing a finger directly on the DBS card and prevents the patient from spotting blood on the wrong side of the DBS card. Further, the device promotes the capillary blood to be collected in manner that is centered on each target spot on the DBS card and additionally prevents for blood to be spotted in other areas of the DBS card. Further yet, the sides of the device have four ventilation openings which allow for more efficient drying. Internal components of the device include small legs throughout the inside of the device that hold the DBS card level and aligned with the collection apertures with minimal contact with the plastic device. The overall device also serves as a protective shield for the DBS card before collection, during collection, and after collection. Further slits or partitions keep blood samples from saturating into adjacent blood samples on the novel DBS cards used in the system. These and other aspects of the subject matter disclosed herein are described below with respect to FIGS. 1-11.

FIG. 1 is an isometric diagram of a dried blood spot (DBS) collection card 100 having a standard form factor for use with an embodiment of the subject matter disclosed herein. Dried blood spot testing (DBS) is a form of bio sampling where blood samples are blotted and dried on filter paper. The dried samples can easily be shipped to a remote analytical laboratory and analyzed using various methods. Dried blood spot specimens are collected by applying a few drops of blood, drawn by lancet (e.g., finger prick) from the finger, heel or toe, onto specially-manufactured absorbent filter paper. The blood is allowed to thoroughly saturate the filter paper at five specific target circles 104a-e and is air dried for several hours. In other embodiment, the number of target spots 104a-e may be more than or less than five. Furthermore, the dimensions of the dried blood spot collection card 100 may be a standard from factor having a length of approximately three inches and a width of five inches, though a skilled artisan understands that this size can be any convenient dimension and the number of spots may be any number including between two and twelve spots.

Once dried, specimens may be stored in low gas-permeability plastic bags with desiccant added to reduce humidity, and may be kept at ambient temperature prior to, during, and after shipping. Once in the laboratory, technicians may separate a small disc of saturated paper (e.g., poke a hole) from the sheet using an automated or manual hole punch, dropping the separated disc into a flat-bottomed microtiter plate. The blood is eluted out from the separated disc into a phosphate buffered saline. The resultant plate containing the eluates forms the “master” from which dilutions can be made for subsequent testing.

In the embodiment shown in FIG. 1, the DBS card 100 includes strategically placed slits 106a-106d that are disposed equidistantly between two respective target spots 104a-104e. The slits 106a-106d are rectangular cutouts when the material that comprises the dried blood spot card 100 filter paper has been removed. That is, there exists a physical separation between target spots so as to assist in maintaining distance and separation between samples on a per-target-spot basis. Thus, one can maintain separation between target-blood-spot samples and, therefore, give rise to opportunities to sample at different times and possibly with different patients. For example, members of one family may each deposit one sample in a specific target-blood-spot for analysis for detection of a specific blood-born illness.

This novel DBS card 100 may be utilized with an objective dried blood collection device (FIG. 2) which is the subject of related U.S. patent application Ser. No. ______ and incorporated herein by reference in its entirety for all purposes. For the remainder of this disclosure, the focus shall remain on the objective dried blood spot collection device as discussed next in FIGS. 2-11.

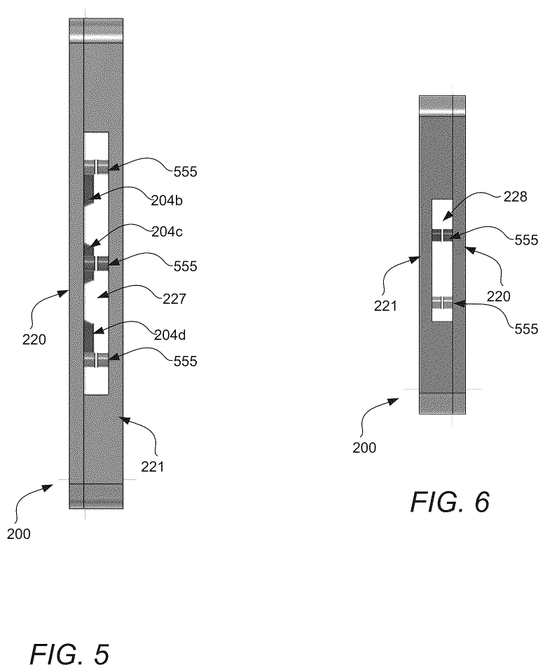

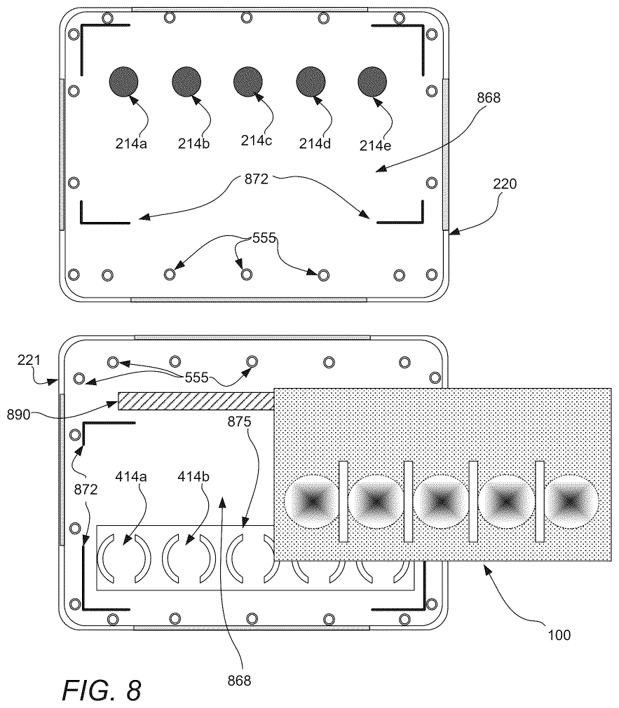

FIG. 2 is an isometric diagram of an objective dried blood spot collection device 200 according to an embodiment of the subject matter disclosed herein. The device 200 is designed to assist with correct and efficient collection of capillary blood onto dried blood spot collection (DBS) card (100 of FIG. 1 but not shown in FIG. 2). The device 200 may comprise a body (comprising top portion 220 and bottom portion 221) having a rectangular shape configured to hold a DBS card 100 in a cavity (800 of FIG. 8 but not shown here). The DBS card 100 is held in the cavity 800 between the top portion 220 and the bottom portion 221 of the device 200 in a desired orientation (e.g., the target circles 104a-104e of FIG. 1 are aligned with the collection apertures 214a-214e as discussed below) for blood sample collection. The top portion 220 and bottom portion 221 are separable and held together in a mating nature using mating portions (555 of FIG. 5 but not shown here) so as to allow access to and removal of the encompassed DBS card 100 when separated. The portions 220 and 221 may be made from an extruded or molded polyurethane plastic having an SPI rating of A2.

The top portion 220 includes five apertures 214a-214e in a top face 223 for assisting in collecting capillary blood from a patient. In embodiments, each aperture comprises a conical opening with an upper diameter (e.g., in the plane of the top face 223) that is between 0.5 to 1.5 inches. The lower diameter is slightly less to provide a conical shape to each aperture 214a-214e. As will be discussed below, the bottom portion 221 also includes a bottom face (224 of FIG. 4) that is unable to be seen in FIG. 2.

When mated, the top portion 220 and bottom portion 221 may exhibit a first elongate ventilation opening 227 along a first direction in two sides (only the one side is shown in FIG. 2) of the overall device 200 that exposes the cavity 800 (where the DBS card 100 is secured) to ambient air. Thus, the first ventilation opening 227 provides a means for assisting in the drying procedure after blood has been collected onto the DBS card 100. Similarly, when mated, the top portion 220 and bottom portion 221 may exhibit a second ventilation opening 228 along a second direction in two sides (only the one side is shown in FIG. 2) of the overall device 200 that exposes the cavity 800 (where the DBS card 100 is secured) to ambient air. As such, the second ventilation opening 228 also provides additional means for assisting in the drying procedure after blood has been collected onto the DBS card 100.

The device 200, as alluded to above, comprises five apertures 214a-214e on the top face 223 of the top portion 220 that are aligned above the circular target spots 104a-104e on the encompassed DBS card 100. When is use, a patient (not shown) will be able to direct drops of capillary blood to pass through the top side 223 such that blood will travel through the apertures 214a-214e to the DBS card 100 at precise locations so as to ensure proper saturation of each respective target spot 104e-104e while minimizing risk of outside contaminants and/or cross-contamination between target spots 104a-104e.

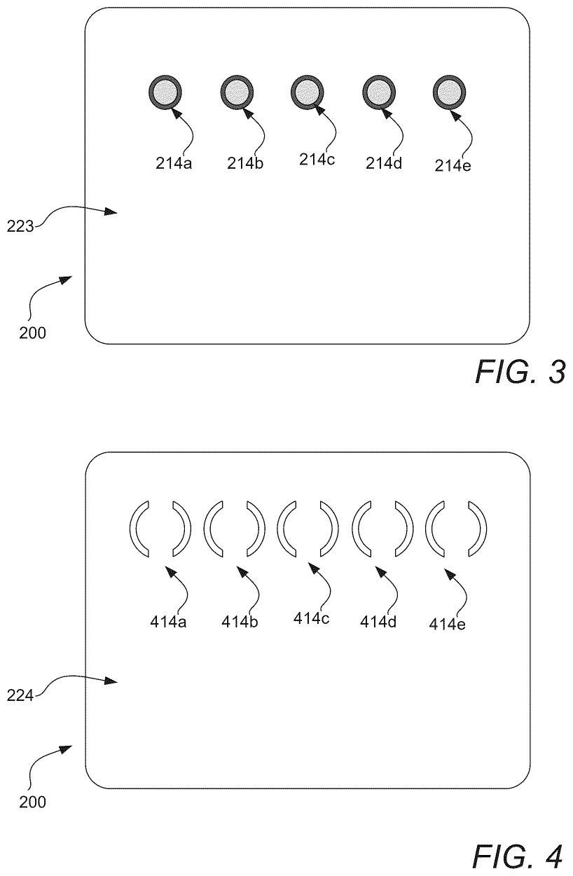

FIG. 3 is a diagram view of the top face 223 of the objective dried blood spot collection device 200 of FIG. 2 according to an embodiment of the subject matter disclosed herein. In this view, the apertures 214a-214e can be seen from a directly overhead view revealing the conical nature of the apertures 214a-214e. Thus, as blood is passed through these apertures 214a-214e, the underlying DBS card 100 may be saturated with blood as needed for the collection procedure.

FIG. 4 is a diagram view of the bottom face 224 of the objective dried blood spot collection device 200 of FIG. 2 according to an embodiment of the subject matter disclosed herein. In this view, observation windows 414a-414e can be seen from a directly overhead view revealing holes resembling crescent shapes of two halves of a circle. Thus, as blood is passed through the top face 223 apertures 214a-214e, the underlying DBS card 100 may be saturated with blood as needed for the collection procedure such that one can see the saturation of the DBS card 100 through the observation windows 414a-414e. This allows the patient to verify saturation level by looking through observation windows 414a-414e disposed on the back face 224 of the device 200. As blood is collected onto the DBS card 100, the ventilation openings 227 and 228 further assist with drying such that the DBS card properly captures blood samples in each of the typical five target spots 104a-104e.

FIG. 5 is a diagram view of one narrow side of the objective dried blood spot collection device 200 of FIG. 2 according to an embodiment of the subject matter disclosed herein. In this view, one can see one of the first ventilation opening 227 on the side of the device 200 formed between the top portion 220 and the bottom portion 221. Through this first ventilation opening 227, one can see three of the five apertures 204b-204d exhibiting a conical shape that opens into the cavity between the top 220 and bottom 221 portions. Further, one can see some of the mating portions 555 between the top 220 and bottom 221 portions.

FIG. 6 is a diagram view of another narrow side of the objective dried blood spot collection device 200 of FIG. 2 according to an embodiment of the subject matter disclosed herein. In this view, one can see one of the second ventilation opening 228 on the side of the device 200 formed between the top portion 220 and the bottom portion 221. Through this first ventilation opening 227, one can also see some of the mating portions 555 between the top 220 and bottom 221 portions. However, the conical apertures 204a-204e cannot be seen through the second ventilation opening 228.

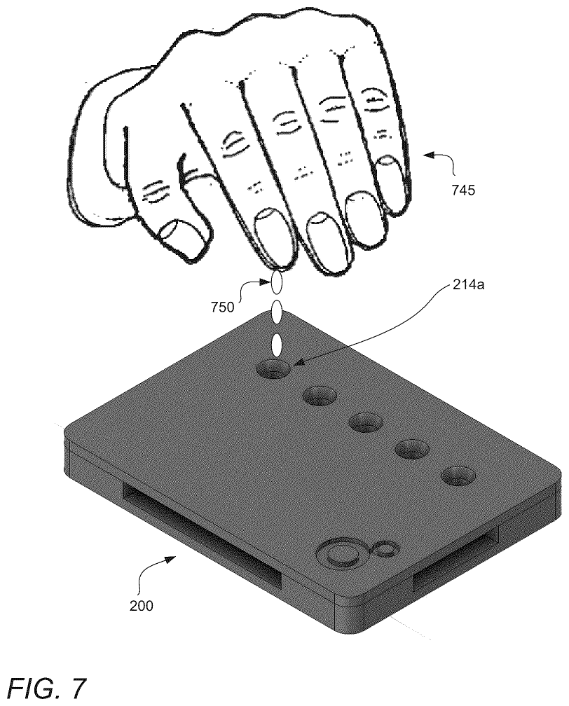

FIG. 7 is a diagram view of the objective dried blood spot collection device 200 of FIG. 2 being used in a process for collecting capillary blood from a human according to an embodiment of the subject matter disclosed herein. In this view, one can see a human hand 745 of a human patient dripping blood 750 (e.g., after being lanced) such that capillary blood 750 may drip down into, in this view, a first collection aperture 214a of the device 200. As the (unseen) DBS collection card 100 becomes saturated, the patient may move the lanced finger on the patient's hand 745 to the next collection aperture for saturated the next target spot on the DBS collection card 100, and so on.

FIG. 8 is a diagram view of the objective dried blood spot collection device 200 of FIG. 2 split into two components (top portion 220 and bottom portion 221) according to an embodiment of the subject matter disclosed herein. This view shows a DBS card 100 being moved into place into the cavity that will be formed by the top portion 220 and bottom portion 221 when mated (e.g., inside the cavity 868). The top portion 220 and bottom portion may be removably secured to each using the mating portions 555 that are distributed around the edges of borders of the cavity 868. In embodiments, these mating portion may comprise mating pegs (for example, the mating portions 555 disposed on inside of the top portion 220) configured to lock into place with matching mating receptables (for example, the mating portion 555 disposed on the inside of the bottom portion 221).

When mated, specific border portions 872 disposed on the insides of both the top portion 220 and the bottom portion may provide a guide for holding the DBS card 100 inside cavity 868 and aligned such that the target blood spots on the DBS card are disposed directly under a respective blood collection aperture 414a-414e and directly over a respective observation window (414a and 414b are shown here).

Additionally, the device 200 may include a desiccant card 890 disposed on the inside of one of the portions. The desiccant card 890 may be adhered to device 200 and aid in dry time but also prevent rejections when patients discard the desiccant pack in a specimen bag or when a kit is incorrectly built without a desiccant for the specimen bag. Having a desiccant may prevent user error and possibly prevent safety issues of a separate desiccant falling out specimen bag or bag such that a person may inadvertently ingest it.

Further yet, the device 200 may include a transparent film 875 disposed on the inside of the bottom portion between the observation windows 414a-414e and the cavity 868 for holding the DBS card 100. The transparent film 875 may be adhered to device 200 and aid in preventing contamination when collected blood soaks through the DBS card and comes into contact with undesired chemicals. Having a transparent film 875 may prevent user error during collection and observation and possibly prevent safety issues of excess blood escaping the collection device exposing others not wishing to be exposed.

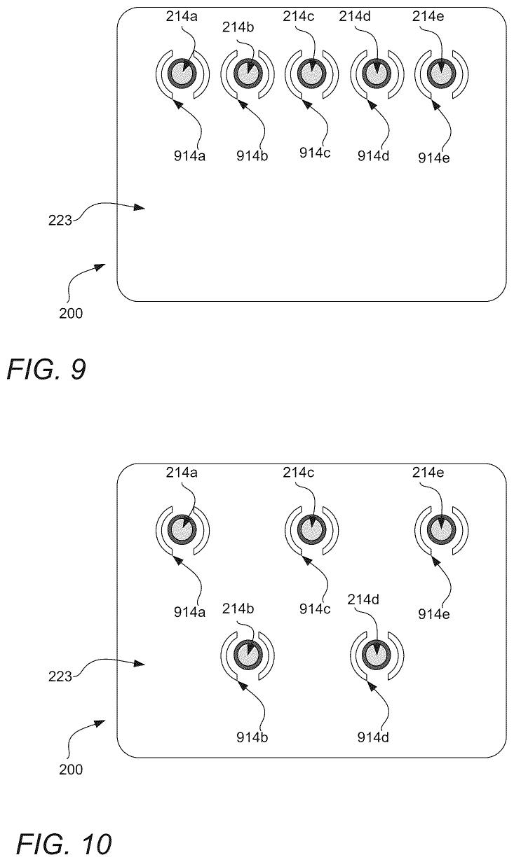

FIG. 9 is a diagram view of another embodiment of the top side of the objective dried blood spot collection device of FIG. 2 according to an embodiment of the subject matter disclosed herein. As was the case from FIG. 2, the device 200 is designed to assist with correct and efficient collection of capillary blood onto dried blood spot collection (DBS) card (100 of FIG. 1 but not shown in FIG. 9). The device 200 may comprise a body (comprising top portion 220 and bottom portion 221) having a rectangular shape configured to hold a DBS card 100 in a cavity 800 (FIG. 8). The DBS card 100 is held in the cavity 800 between the top portion 220 and the bottom portion 221 of the device 200 in a desired orientation (e.g., the target circles 104a-104e of FIG. 1 are aligned with the collection apertures 214a-214e as discussed below) for blood sample collection. The top portion 220 includes five apertures 214a-214e in a top face 223 for assisting in collecting capillary blood from a patient. In embodiments, each aperture comprises a conical opening with an upper diameter (e.g., in the plane of the top face 223) that is between 0.5 to 1.5 inches. The lower diameter is slightly less to provide a conical shape to each aperture 214a-214e.

In this view, the apertures 214a-214e can be seen from a directly overhead view revealing the conical nature of the apertures 214a-214e. Thus, as blood is passed through these apertures 214a-214e, the underlying DBS card 100 may be saturated with blood as needed for the collection procedure.

Further, in this view, front-facing observation windows 914a-914e can be seen from a directly overhead view of the top-side revealing holes resembling crescent shapes of two halves of a circle. Each respective front-facing observation windows 914a-914e encircles a respective aperture 214a-214e. Thus, as blood is passed through the top face 223 apertures 214a-214e, the underlying DBS card 100 may be saturated with blood as needed for the collection procedure such that one can see the saturation of the DBS card 100 through the observation windows 914a-914e. This allows the patient to verify saturation level by looking through front-facing observation windows 914a-914e disposed on the top face 223 of the device 200 such that the patient does not need to flip the device 200 over to verify saturation levels. The front-facing observation windows 914a-914e may also include transparent window materials to prevent blood from entering the front-facing observation windows 914a-914e. This provides a way to observe through to the DBS card at the edges of the target spots 104a-104e but without risk of contamination through the front-facing observation windows 914a-914e.

FIG. 10 is a diagram view of yet another embodiment of the top side 223 of the objective dried blood spot collection device 200 of FIG. 2 according to an embodiment of the subject matter disclosed herein. As was the case from FIG. 2, the device 200 is designed to assist with correct and efficient collection of capillary blood onto dried blood spot collection (DBS) card (100 of FIG. 1 but not shown in FIG. 9). The device 200 may comprise a body (comprising top portion 220 and bottom portion 221) having a rectangular shape configured to hold a DBS card 100 in a cavity 800 (FIG. 8). The DBS card 100 is held in the cavity 800 between the top portion 220 and the bottom portion 221 of the device 200 in a desired orientation (e.g., the target circles 104a-104e of FIG. 1 are aligned with the collection apertures 214a-214e in this “W” pattern as discussed below) for blood sample collection. The top portion 220 includes five apertures 214a-214e in a top face 223 arranged in a “W” pattern for assisting in collecting capillary blood from a patient. In embodiments, each aperture comprises a conical opening with an upper diameter (e.g., in the plane of the top face 223) that is between 0.5 to 1.5 inches. The lower diameter is slightly less to provide a conical shape to each aperture 214a-214e. Having a “W” pattern provides for additional separation between apertures to further assist with preventing cross-contamination between target spots.

In this view, the apertures 214a-214e can be seen from a directly overhead view revealing the conical nature of the apertures 214a-214e as well as the “W” pattern of alignment. Thus, as blood is passed through these apertures 214a-214e, the underlying DBS card 100 may be saturated with blood as needed for the collection procedure.

Further, in this view, front-facing observation windows 914a-914e can be seen from a directly overhead view of the top-side 223 revealing holes resembling crescent shapes of two halves of a circle. Each respective front-facing observation windows 914a-914e encircles a respective aperture 214a-214e. Thus, as blood is passed through the top face 223 apertures 214a-214e, the underlying DBS card 100 may be saturated with blood as needed for the collection procedure such that one can see the saturation of the DBS card 100 through the observation windows 914a-914e. This allows the patient to verify saturation level by looking through front-facing observation windows 914a-914e disposed on the top face 223 of the device 200 such that the patient does not need to flip the device 200 over to verify saturation levels. The front-facing observation windows 914a-914e may also include transparent window materials to prevent blood from entering the front-facing observation windows 914a-914e. This provides a way to observe through to the DBS card at the edges of the target spots 104a-104e but without risk of contamination through the front-facing observation windows 914a-914e.

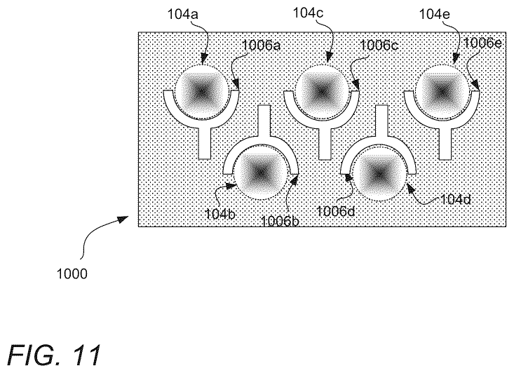

FIG. 11 is a top-view diagram of another dried blood spot collection card 1000 for use with an objective dried blood spot collection device according to an embodiment of the subject matter disclosed herein. As before, dried blood spot specimens are collected by applying a few drops of blood, drawn by lancet (e.g., finger prick) from the finger, heel or toe, onto specially-manufactured absorbent filter paper. The blood is allowed to thoroughly saturate the filter paper at five specific target circles 104a-e and is air dried for several hours. As can be seen in this embodiment, the five specific target circles 104a-e are arranged in a “W” pattern such that three target circles 104a, 104c, and 104e are disposed in line across an upper portion of the card 1000 while two other target circles 104b and 104d are disposed in a line across a lower portion of the card 1000. In other embodiments, the number of target spots 104a-e may be more than or less than five. Furthermore, the dimensions of the dried blood spot collection card 1000 may be a standard from factor having a length of approximately three inches and a width of five inches, though a skilled artisan understands that this size can be any convenient dimension and the number of spots may be any number including between two and twelve spots.

Once dried, specimens may be stored in low gas-permeability plastic bags with desiccant added to reduce humidity, and may be kept at ambient temperature prior to, during, and after shipping. Once in the laboratory, technicians may separate a small disc of saturated paper (e.g., poke a hole) from the sheet using an automated or manual hole punch, dropping the separated disc into a flat bottomed microtiter plate. The blood is eluted out from the separated disc into a phosphate buffered saline. The resultant plate containing the eluates forms the “master” from which dilutions can be made for subsequent testing.

In the embodiment shown in FIG. 11, the DBS card 1000 includes strategically placed partitions 1006a-1006d that are regions of the DBS card that have been removed. That is, similar to the slits 106a-106e of FIG. 1, the material where partitions 1006a-1006d of the DBS card 1000 has been removed such that blood will not saturate the paper beyond the slits when deposited. In this embodiment, partitions 1006a-1006d are disposed partially surrounding each respective target spots 104a-104e. The partitions 1006a-1006d also include respective rectangular cutouts below a crescent portion when the material that comprises the dried blood spot card 1000 filter paper has been removed. This specific partition shape enables physical separation between target spots so as to assist in maintaining distance and separation between samples on a per-target-spot basis in a “W” shaped dried blood spot collection system.

The use of the terms “a” and “an” and “the” and similar referents in the specification and in the following claims are to be construed to cover both the singular and the plural, unless otherwise indicated herein or clearly contradicted by context. The terms “having,” “including,” “containing” and similar referents in the specification and in the following claims are to be construed as open-ended terms (e.g., meaning “including, but not limited to,”) unless otherwise noted. Recitation of ranges of values herein are merely indented to serve as a shorthand method of referring individually to each separate value inclusively falling within the range, unless otherwise indicated herein, and each separate value is incorporated into the specification as if it were individually recited herein. All methods described herein can be performed in any suitable order unless otherwise indicated herein or clearly contradicted by context. The use of any and all examples, or exemplary language (e.g., “such as”) provided herein, is intended merely to better illuminate embodiments and does not pose a limitation to the scope of the disclosure unless otherwise claimed. No language in the specification should be construed as indicating any non-claimed element as essential to each embodiment of the present disclosure.

Different arrangements of the components depicted in the drawings or described above, as well as components and steps not shown or described are possible. Similarly, some features and sub-combinations are useful and may be employed without reference to other features and sub-combinations. Embodiments have been described for illustrative and not restrictive purposes, and alternative embodiments will become apparent to readers of this patent. Accordingly, the present subject matter is not limited to the embodiments described above or depicted in the drawings, and various embodiments and modifications can be made without departing from the scope of the claims below.

That which is claimed is:

1. A collection card for collecting dried blood spots for diagnostics, comprising:
a card body comprising absorbent filter paper configured to receive one or more blood samples;
a plurality of blood collection targets disposed in a pattern on a top face of the card body; and
a plurality of openings disposed in a pattern that provides separation between adjacent blood collection targets, the plurality of opening characterized by a lack of absorbent filter paper.

2. The collection card of claim 1, wherein the plurality of blood collection targets comprises five blood collection targets.

3. The collection card of claim 1, wherein the pattern of the plurality of blood collection targets comprises blood collection targets disposed equidistantly in a single line.

4. The collection card of claim 1, wherein pattern of the plurality of blood collection targets comprises blood collection targets disposed in a “W” pattern.

5. The collection card of claim 1, further comprising a form factor configured to placed inside an objective dried blood spot collection device.

6. The collection card of claim 1, further comprising a desiccant.

7. The collection card of claim 1, wherein the plurality of openings further comprise elongated rectangular shapes disposed equidistantly between adjacent blood collection targets.

8. The collection card of claim 1, wherein the plurality of openings further comprise crescent shapes disposed adjacent to a respective blood collection target.

9. The collection card of claim 8, wherein the plurality of openings further comprise a respective rectangular opening disposed intersecting a respective crescent shape.

10. The collection card of claim 1, wherein the plurality of blood collection targets comprises between two and twelve blood collection targets.

11. A system for facilitating capillary blood collection, the system comprising:
a dried blood spot collection card including:
a card body comprising absorbent filter paper configured to receive one or more blood samples;
a plurality of blood collection targets disposed in a pattern on a top face of the card body; and
a plurality of openings disposed in a pattern that provides separation between adjacent blood collection targets, the plurality of opening characterized by a lack of absorbent filter paper; and
a dried blood spot collection device, including:
a body having a first portion and second portion coupled to each other and encapsulating a cavity configured to hold the dried blood spot collection card;
a plurality of blood collection apertures configured to direct blood toward the dried blood spot collection card; and
a plurality of observation windows disposed configured to provide a view to the dried blood spot collection card.

12. The system of claim 11, wherein the plurality of blood collection target spots corresponds to the plurality of blood collection apertures and corresponds to the plurality of observation windows.

13. The system of claim 11, wherein the dried blood spot collection card further comprises five blood spot targets disposed in a “W” pattern with respect to each other.

14. The system of claim 11, wherein the dried blood spot collection card further comprises five blood spot targets disposed in a row adjacent to each other.

15. The system of claim 11, further comprising a desiccant.

16. The system of claim 11, wherein the plurality of blood collection apertures comprises five blood collection apertures disposed in a “W” pattern.

17. The system of claim 11, wherein the plurality of observation windows comprises a number equivalent to the plurality of blood collection apertures.

18. The system of claim 11, wherein the first portion further comprises a plurality of mating pegs disposed on a side of the first portion that is opposite the top face;
wherein the second portion further comprises a plurality of mating receptacles disposed on a side of the second portion that is opposite the bottom face; and
wherein respective mating pegs are configured to mate with respective mating receptacles to removably secure the first portion to the second portion.

19. The system of claim 11, further comprising a transparent material disposed between the dried blood collection card and the plurality of observation windows.

20. The system of claim 11, further comprising plastic portions having extruded polyurethane with an SPI-A2 rating.
